(19) United States
(12) Patent Application Publication
(10) Pub. No.: US 2007/0148141 A1
(43) Pub. Date: Jun. 28, 2007

(54) METHOD OF USING HEPATIC PROGENITORS IN TREATING LIVER DYSFUNCTION

(75) Inventors: Mark Johnston, Durham, NC (US); John Ludlow, Durham, NC (US); Sonya Sherwood, Durham, NC (US)

Correspondence Address: FOLEY AND LARDNER LLP;SUITE 500, 3000 K STREET NW, WASHINGTON, DC, 20007, US

(73) Assignee: Vesta Therapeutics Inc.

(21) Appl. No.: 11/614,703

(22) Filed: Dec. 21, 2006

Related U.S. Application Data
(60) Provisional application No. 60/752,597, filed on Dec. 22, 2005.

(51) Int. Cl. A61K 35/407

(52) U.S. Cl. 424/93.7

(57) ABSTRACT

Methods of using hepatic progenitors in treating liver dysfunction are provided. More particularly, methods of using hepatic progenitor cells, including hepatic stem cells, in treating liver dysfunction in the absence of immune-suppressing amounts of an immunosuppressant.

### CROSS REFERENCES TO RELATED PATENT APPLICATIONS

This application claims priority to U.S. Provisional Application No. 60/752,597, filed Dec. 22, 2005, the disclosure of which has been incorporated herein by reference in its entirety.

### FIELD OF THE INVENTION

The present invention relates generally to the use of using hepatic progenitors in treating liver dysfunction. More particularly, the present invention relates to methods of using hepatic progenitor cells, including hepatic stem cells, in treating liver dysfunction without concomitant use of immunosuppressants.

### BACKGROUND OF THE INVENTION

The efficacy of whole and partial liver transplants is substantially dependent on the host's immune response (e.g., Graft versus Host Disease (“GVHD”)). Indeed, among genetically non-identical patients, the host's body may reject donor tissue in a matter of hours. To account for this potential, substantial time and effort is exhausted to genetically “match” donors with possible hosts.

In the absence of genetically identical twins, however, the immune system of even the best “matched” recipient will nonetheless mount a response to reject the donor tissue. Thus, nearly all organ transplants are followed by substantial use of immunosuppressants in the short term, if not for the duration of the transplant. With respect to liver transplants, for example, the calcineurin inhibitors, tacrolimus and cyclosporine, are widely used to prevent allograft rejection.

Unfortunately, immunosuppressants are not a panacea. First, immunosuppressants are not always affective and must be taken on a regular schedule. Second, chronic use of these drugs leaves a patient vulnerable to bacterial and viral infections that can further aggravate their condition. Indeed, calcineurin inhibitors can lead to in nephrotoxicity, often resulting in prolonged renal failure after transplantation. Additional adverse long-term side effects include glucose intolerance and hyperlipidemia. Use of immune privileged cells, therefore, would reduce the need, if any, for immunosuppression and their corresponding adverse side effects.

Accordingly, there is a need for a method of treating liver dysfunction with allogenic cells that do no illicit an immune response. There is also a need for a method of transplanting hepatic cells with little to no immunosuppresants.

### SUMMARY OF THE INVENTION

In one embodiment of the present invention, a method of repopulating the liver compartment of a mammal is provided, comprising (a) providing isolated hepatic progenitors and (b) administering said isolated hepatic progenitors into the liver compartment of the mammal; wherein the administering is performed with low levels of immunosuppressants. The administration may be by implant or injection. Further, the low level of immunosuppressant ranges between about 0 mg to about 10 mg. More preferably, the low level of immunosuppressant ranges between about 0 mg to about 5 mg.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 shows the response of unfractionated PBMCs to identified liver cell populations.

FIG. 2 shows the response of purified T cells to identified liver cell populations.

FIG. 3 shows the results from mixed lymphocyte reaction assays performed on the identified cell populations. 3A: PBMC response. 3B: Purified T-cell response.

FIG. 4 shows the purified T cell response to EpCAM positive liver cells.

FIG. 5 shows another purified T cell response to EpCAM positive liver cells.

FIG. 6 shows yet another purified T cell response to EpCAM positive liver cells.

FIG. 7 shows further yet another purified T cell response to EpCAM positive liver cells.

FIG. 8 shows the suppression of a mixed lymphocyte reaction by EpCAM positive cells.

### DETAILED DESCRIPTION OF THE INVENTION

The instant invention provides for methods of transplanting hepatic progenitor cells into mammals without the need of currently standard doses of immunosuppressants, if any at all. That is, in a preferred embodiment of the invention, hepatic progenitors may be introduced into an allogenic host with little to no concomitant administration of an immunosuppressants to prevent the rejection thereof.

Currently, most, if not all, partial or whole liver transplants into an allogeneic recipient requires the use of an immunosuppressant in order to prevent immune rejection (i.e., GVHD) of the transplanted tissue. While the dose and duration of immunosuppresant use varies greatly among transplants according to factors, such as tissue to, relative genetic “match”, the instant discovery and invention provides a significant reduction in the overall usage of the drugs. Common immunosuppressants include, but are not limited to consisting of prednisone (e.g., ORASONE®), azathioprine (e.g., IMURAN®), cyclosporine (e.g., SANDIMMUNE®, NEORAL®), and mycophenolate (e.g., CELLCEPT®).

Currently, common immunosuppressant doses range from about 5 mg to about 20 mg. See Table 1, below. The instant invention provides for allogeneic transplantation of liver cells at sub-immune suppressing amounts of immunosuppressant doses, and preferably no immunosupressants at all. In other words, for any individual recipient, a sub-immune suppressing amount of an immunosuppressant is defined as an amount less than that which would give rise to a suppression of that individual's immune response. Assays are available and known to those of ordinary skill in the art to measure the optimal dose of an immunosuppressant to achieve immunosuppression. These tests include, for example, quantitative radioreceptor assays, scintillation proximity assays, MTT assays and commercially available clinical kits and analyzers (e.g., IMx® Sirolimus assay using Microparticle Enzyme Immunoassay (MEIA) technology from Abbott Labs; CEDIA CsA PLUS from Roche Diagnostics. As well, in vitro assays such as those described below can also provide reasonable correlations to effective dose curves in vivo. As defined in the present application, a dose response of less than about 30% of the optimal dose response is considered sub-immune, preferably a dose response of less than about 60%, and more preferably a dose response of less than about 90%. Typically, sub-immune suppressing doses of the present invention preferably range from about 0 mg to about 10 mg, more preferably from about 0 mg to about 5 mg, and most preferably from about 0 mg to about 3 mg.

TABLE 1
General guidelines for long-term prednisone dosage and regimens
for tapering recipients off prednisone.

| Time post-transplant | Dosage (mg) |
|---|---|
| Tacrolimus-based immunosuppression | |
| 0-2 weeks | 20 |
| 2-8 weeks | 15 |
| 8-16 weeks | 10 |
| 4-6 months | 7.5 |
| 6-9 months | 5 |
| >9 months | Taper |
| | and discontinue |
| Tacrolimus in patients with chronic hepatitis B or C | |
| 0-2 weeks | 20 |
| 2-4 weeks | 15 |
| 4-8 weeks | 10 |
| 2-3 months | 7.5 |
| 3-6 months | 5 |
| >6 months | Taper |
| | and discontinue |
| Cyclosporine-based immunosuppression | |
| 0-2 weeks | 20 |
| 2-26 weeks | 15 |
| 6-9 months | 10 |
| 9-12 months | 7.5 |
| 12-18 months | 5.0 |

(See http ://med.stanford.edu/shs/txp/livertxp/outpatient.management/management.html#t3.)

The invention has utility in repopulating diseased or dysfunctional liver tissue with otherwise healthy donor tissue cells. Repopulation of a diseased or dysfunction liver with functional liver tissue can replace, in at least some part, the activity lost from the malfunctioning tissue. In this way, diseases of the liver may be treated.

The invention will now be described in non-limiting examples.

Isolation of Human Cells

It is noted that hepatic progenitors suitable for in vitro propagation in accordance with the instant invention are not limited to those isolated or identified by any particular method. By way of example, methods for the isolation and identification of the hepatic progenitors have been described in, for example, U.S. Pat. No. 6,069,005 and U.S. patent application Ser. Nos. 09/487,318; 10/135,700; and 10/387,547, the disclosures of which are incorporated herein in their entirety by reference.

Hepatic stem cells and hepatoblasts have characteristic antigenic profiles and can be isolated by protocols described previously. For example, hepatic stem cells and hepatoblasts share numerous antigens (e.g., cytokeratins 8, 18, and 19, albumin, CD133/1, and epithelial cell adhesion molecule (“EpCAM”) and are negative for hemopoietic markers (e.g., glycophorin A, CD34, CD38, CD45) and mesenchymal cell markers (e.g., CD146). Alternatively, hepatic stem cells and hepatoblasts can be distinguished from each other by size (the stem cells are 7-9 μm; the hepatoblasts are 10-12 μm) or by antigenic profiles (N-CAM is present in the hepatic stem cells, whereas alpha-fetoprotein (AFP) and ICAM1 are expressed by the hepatoblasts).

In the instant Example, donated livers, not suitable for orthotopic liver transplantation, were obtained from federally designated organ procurement organizations. Informed consent was obtained from next of kin for use of the livers for research purposes. To isolate cells, adult and pediatric livers were perfused through the portal vein and hepatic artery with EGTA-containing buffer for 15 min followed by 125 mg/L Liberase (Roache) for 30 min at 34° C. The cells were passed sequentially through filters of pore size 1,000, 500, 250, and 150 microns, and centrifuged in density gradients at 500×g.

Magnetic Cell Selection

Magnetic cell selection can offer a convenient way to isolate cells expressing a specific surface antigen from a heterogeneous cell population. In one embodiment, a commercially available system (“MACS” MicroBeads, Miltenyi Biotec) was used to perform the task of selecting for cells positive for EpCAM, a marker indicative of hepatic progenitor cells. A monoclonal antibody to the antigen of interest can be coupled to super paramagnetic microbeads, composed of iron oxide and a polysaccharide coat. The microbeads are approximately 50 nanometers in diameter and have a volume about one-millionth that of a typical mammalian cell. They are small enough to remain in colloidal suspension, which can permit rapid, efficient antibody-mediated binding to cell surface molecules. The manufacturers have provided evidence that the microbeads do not interfere with flow cytometry, that they are biodegradable, and that they have negligible effects on cellular functions.

The antibody used for selection can be used in a direct method, as described above, or an indirect one in which the beads are covalently bound to a monoclonal antibody against a ligand such as fluorescein or biotin and then utilized to capture cells with a surface bound monoclonal antibody that has been modified with that ligand. With either direct or indirect immunoselection methods, cells that have been incubated with the labeled beads are passed through a column in the presence of a strong magnetic field so that antigen-positive cells are retained while negative cells are washed through. After removal of the magnetic field, the positively labeled cells are collected.

FACs Analyses for Antigenic Markers

For cell surface markers (e.g.: EpCAM), 1×10⁶ cells were pelleted at 500×g for 5 min. and resuspended in 100 μL PBS containing 2% FBS and 0.01% NaN₃. 1 μg of the antibody of interest was then added and incubated for 30 min. on ice in the dark. Cells were then washed 2 times with PBS and the final cell pellet resuspended in 0.5 ml of 1% paraformaldehyde. 1 μg mouse IgG1 FITC and mouse IgG1 PE were added to appropriate control cells.

Mixed Lymphocyte Reaction for Immunogenicity

A mixed lymphocyte reaction (MLR) assay was used to assess relative immunogenicity in vitro. This assay measures the proliferation of responder T cells to an allogeneic stimulator population. Briefly, human peripheral blood mononuclear cells (PBMCs) were purified from whole blood by centrifugation over ficoll/hypaque. These cells, as well as T cells isolated from the PBMC population, were used as responder cells in the MLR. T cells were enriched by negative selection techniques by tagging B cells and monocytes with antibody-coated magnetic beads and removing them with a magnet. Stimulator cells were freshly isolated liver cells and isolated hepatic progenitor cells.

Each stimulator cell population was treated with mitomycin C (10 μg/ml mitomycin C for 3 hr.) or irradiated to prevent stimulator cells from proliferating in the assay. Responder T cells from an individual other than the donor (leukopacks were purchased from Poietics, Inc.) were cultured (2×10⁵ cells/well) with varying numbers of each stimulator population (range 0.2-2.0×10⁵ cells/well) in tissue culture medium supplemented with 5% human AB serum in 96 well flat-bottomed plates. Additional control cultures consisted of responder or stimulator cells cultured alone. Quadruplicate cultures were set up for each treatment. Cultures were incubated at 37° C. in a humidified, 5% CO₂-containing atmosphere for 6 days. The cultures were then pulsed with 1 μCi/well 3H-thymidine for 16 hr., harvested onto glass fiber filters, and counted in a scintillation counter.

The amount of incorporated radioactivity (cpm) was presented a proportional to T cell proliferation. Delta cpms were calculated based on the T cell response to a stimulator population, subtracting the appropriate background of the responder and stimulator populations cultured alone.

Mixed Lymphocyte Reaction for Immunosuppression

The MLR assay, essentially as described above, was also performed using the allogeneic stimulator cell population. To these reactions, non-suppressive splenic fibroblasts (5×10³, 10×10³, and 20×10³ cells) were titrated as controls. Parallel reactions with freshly isolated progenitor cells were titrated in “experimentals”. ³H-thymidine incorporation into T cells was then measured. Experimental levels of ³H-thymidine incorporation equal to or greater than those of the controls were interpreted to mean that the hepatic progenitor cells are not capable of suppressing an immune response.

Results

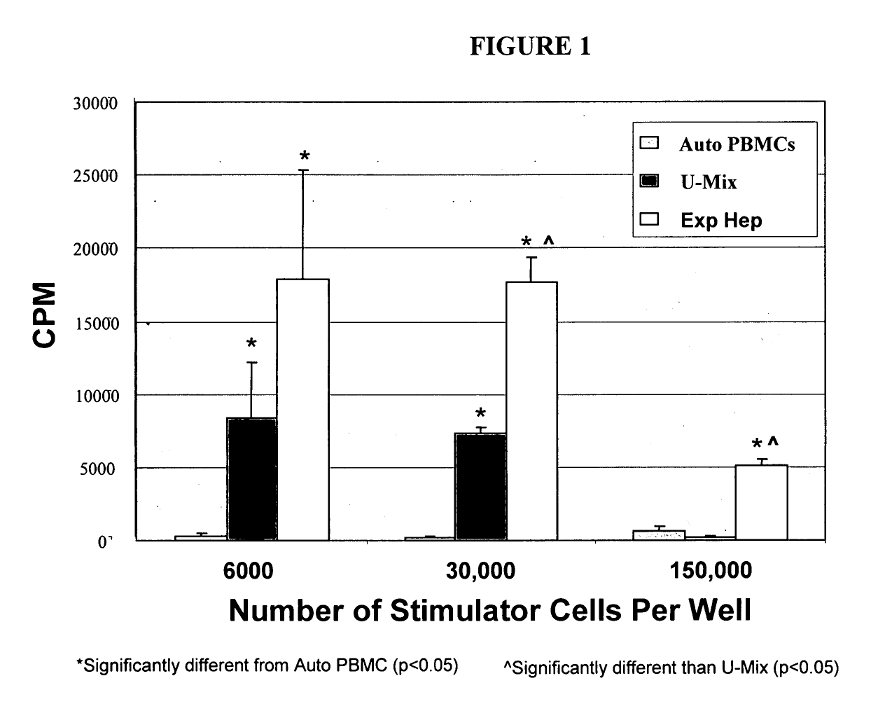

An unfractionated mixture of liver cells (“Umix”) and an adherent population derived from a 3-day culture of Umix cells (Expanded Hepatocytes”) were studied. As shown in FIG. 1, unfractionated PBMCs responded significantly (P<0.05) above autologous background responses to irradiated Umix and Expanded Heptocytes at does levels of 6,000/well, 30,000/well, and 150,000/well (Expanded Hepatocytes only). Moreover, the response to Expanded Hepatocytes was significantly higher for all doses than the response to Umix cells (p<0.05). Responses at the highest dose of 150,000 liver cells/well were low, most likely due to crowding (Umix cells and Expanded Hepatocytes are large cells that completely cover the wells of the 30,000 cells/well dose). The lowest dose of stimulatory cells, both populations of liver cells were more stimulatory than allogeneic PBMCs. At higher doses, PBMCs were more stimulatory.

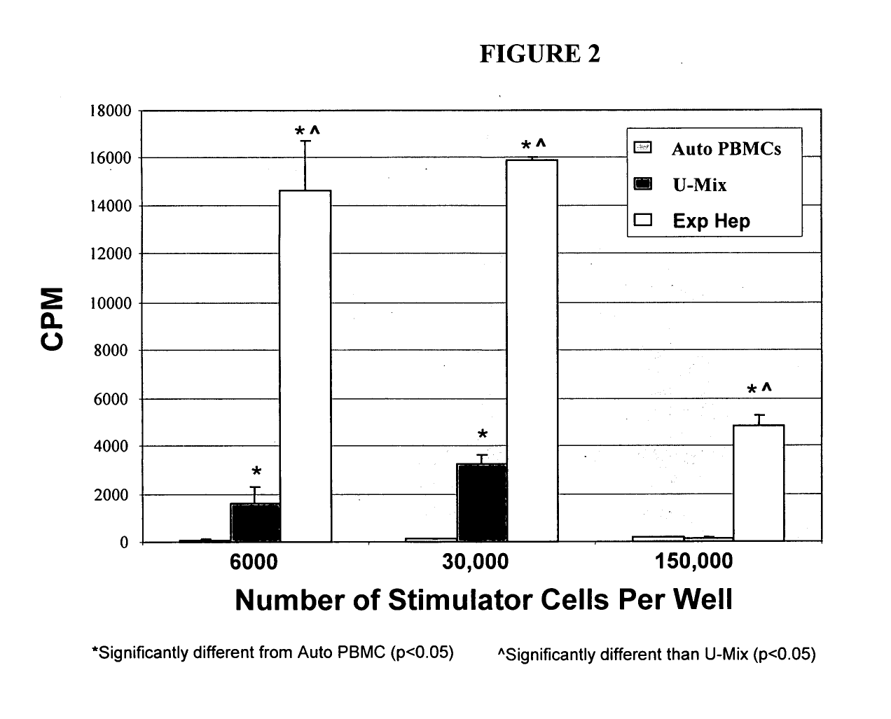

As shown in FIG. 2, purified T cells gave a similar pattern of responses to Umix and Expanded Hepatocytes. Expanded Hepatocytes significantly stimulated T cells above background levels at all doses and the response to the expanded population was significantly higher than the response to Umix cells. Purified T-cells (deficient in antigen presenting cells (APCs)) responded vigorously to Expanded Hepatocytes suggesting that these cells could function as APCs. T cells produced a less vigorous response to the Umix population than PBMC responder cells (containing APCs) suggesting that an exogenous source of APCs was required for this response.

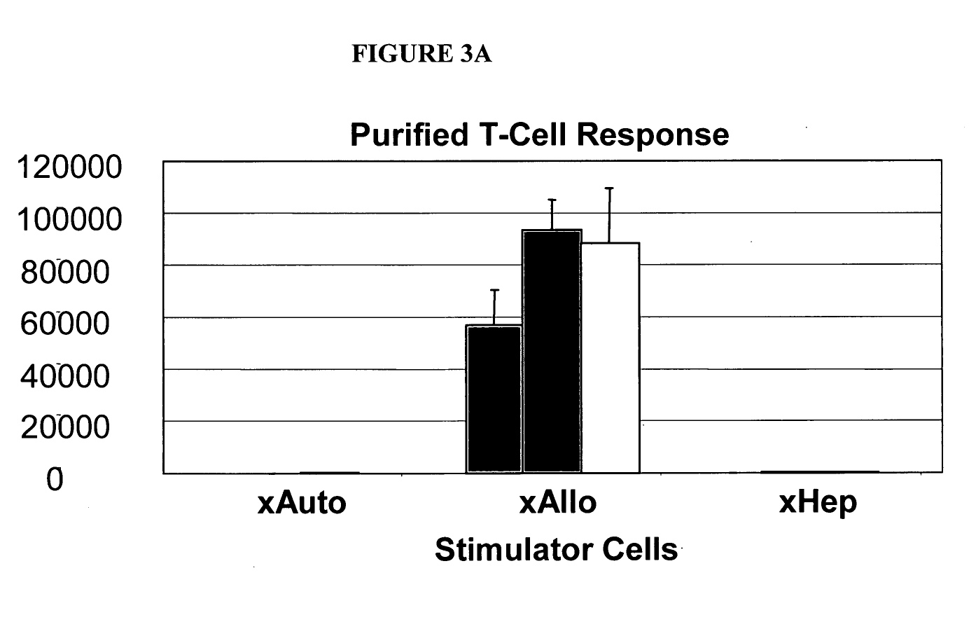

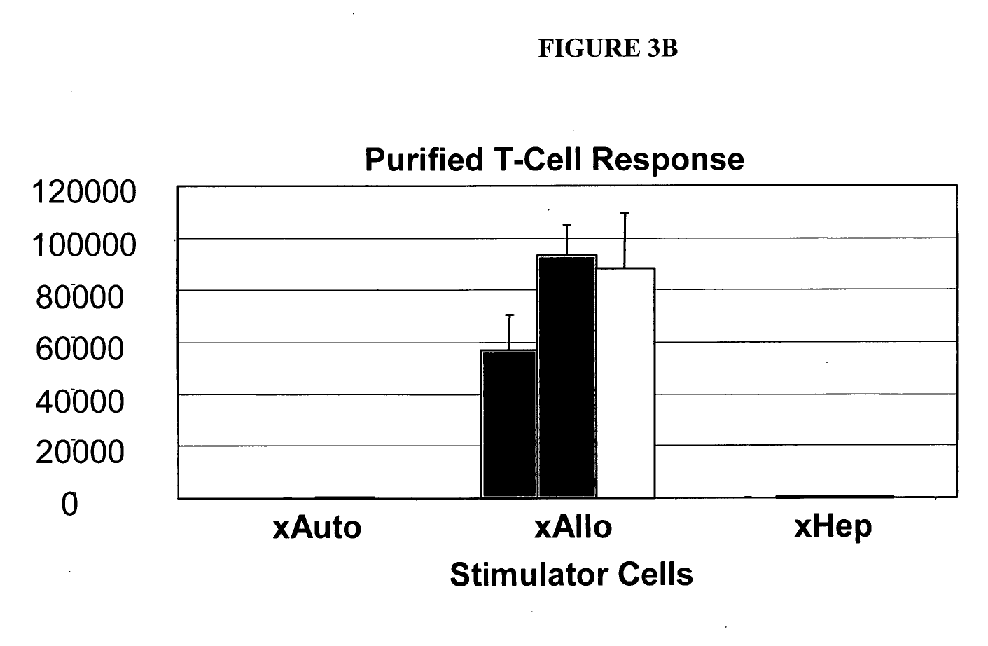

Similar experiments were first performed on adult hepatic stem/progenitor cells immunoselected with antibody to EpCAM and isolated by magnetic activated cell sorting. FACs analysis of the isolated cell population revealed a purity of about 82%. The results of the MLR experiment on these cells are shown in FIG. 3. Responder cells consisting of peripheral blood mononuclear cells (PMBCs, panel A; 3×10⁵ cells) and purified T cells (panel B; 2×10⁵ cells) were mixed with stimulator cells comprised of autologous PMBCs (xAuto), allogeneic PMBCs (xAllo), or adult hepatic progenitor cells (xHep). Different amounts of these stimulator cells were tested (blue bar=12.5×10³ cells; purple bar=25×10³ cells; yellow bar=50×10³ cells).

The allogeneic PMBCs elicited an immune response, as detected by increased ³H-thymidine uptake of the proliferative T cell population. In contrast, the magnitude of T cell proliferation to the adult hepatic progenitor cells was not significantly different from the response to autologous control PBMCs. These data support the conclusion that the adult hepatic stem/progenitor cells are not immunogenic.

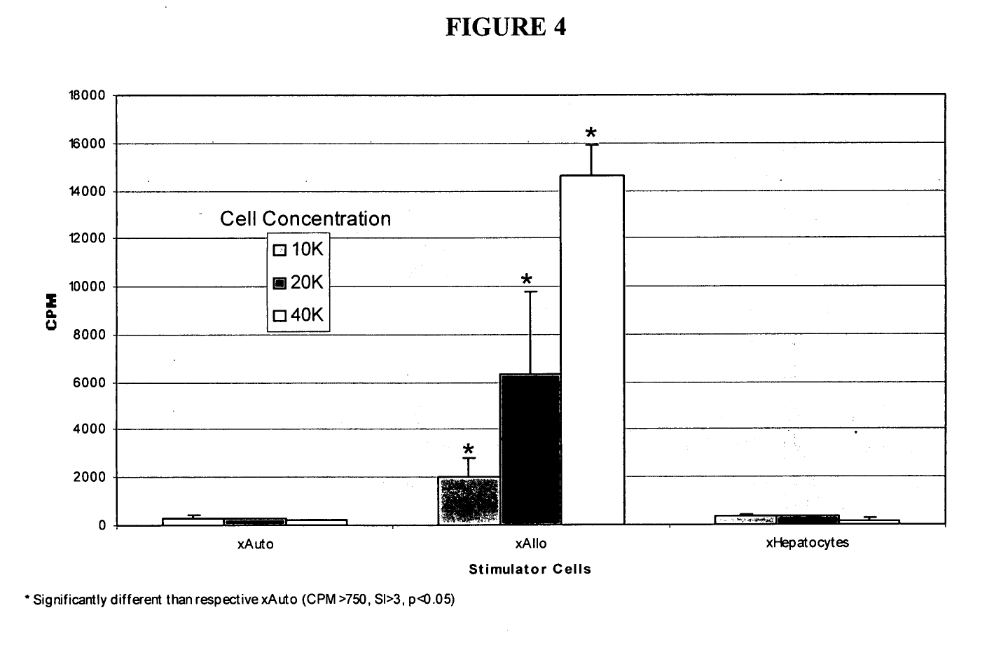

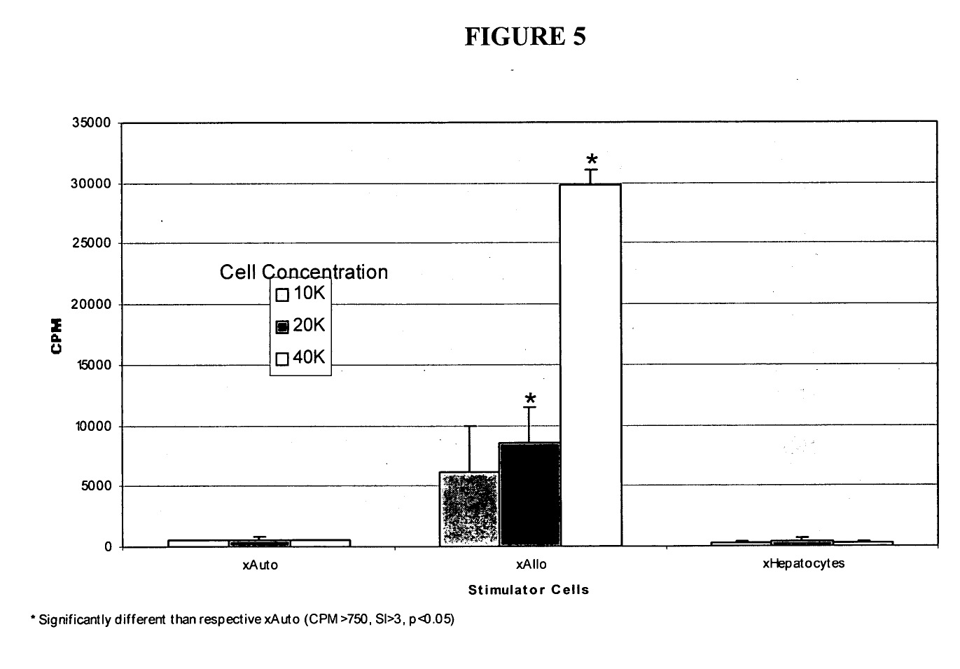

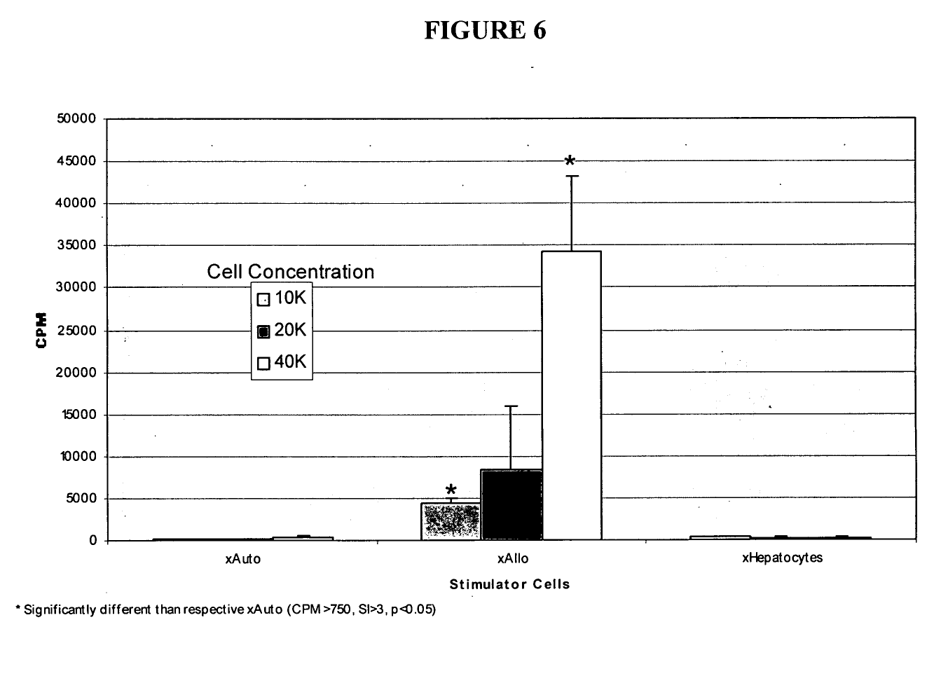

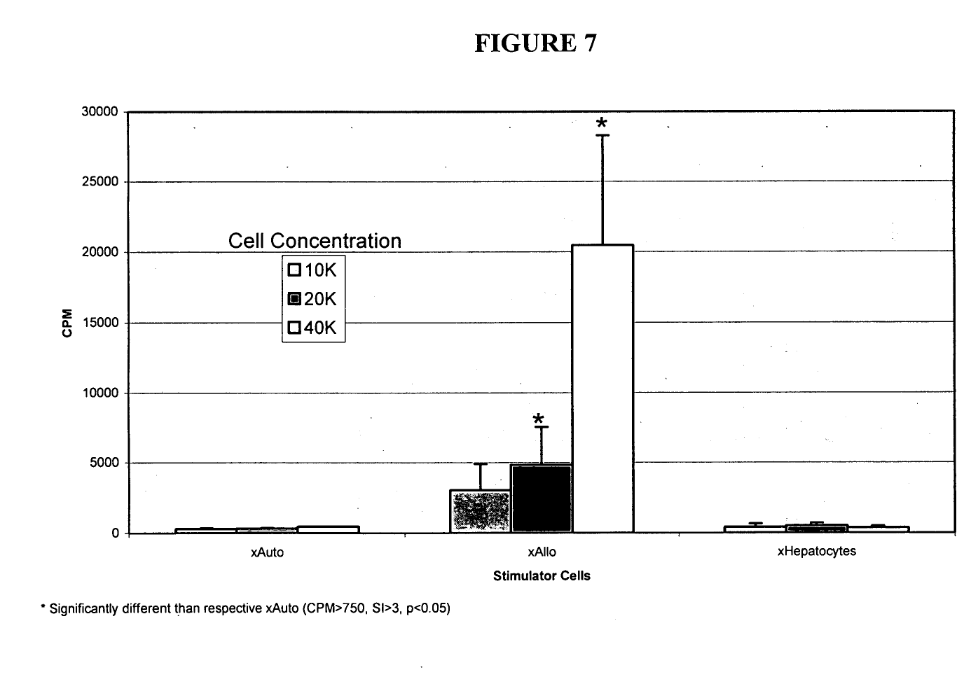

Using a different preparation of adult hepatic stem/progenitor cells, an experiment to evaluate the immunogenicity of these cells was performed. Additional experiments to evaluate the immunosuppression ability of these cells were also carried out. FIGS. 4-7 reveal that the EpCAM+ cells did not stimulate a T cell proliferative response from either one of the two different PMBC and T cell donors.

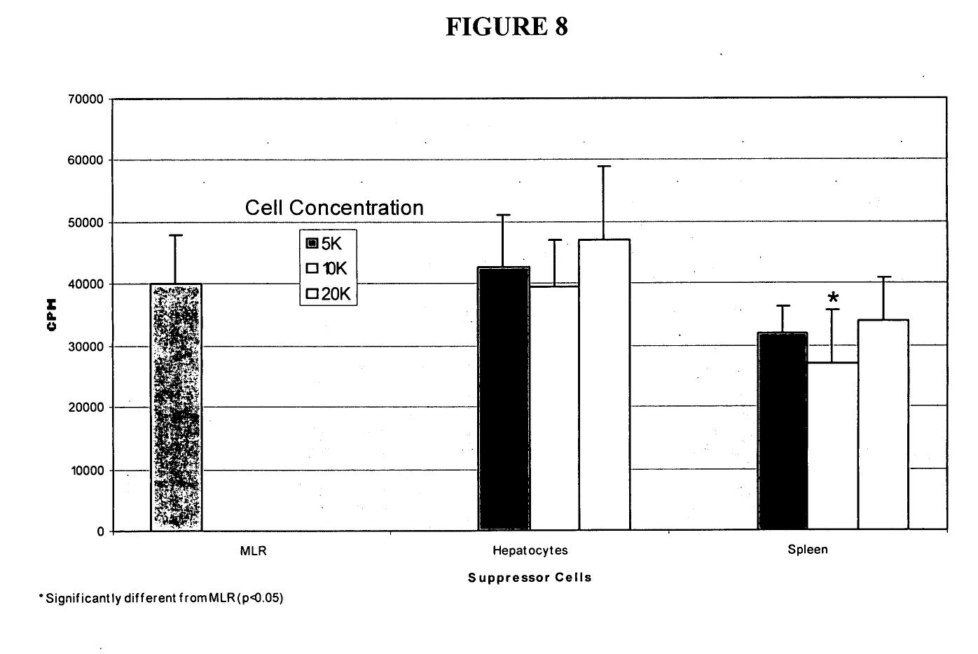

Results from the immunosuppression experiments are shown in FIG. 8. The data reveal that the adult hepatic stem/progenitor cells did not suppress the MLR response. The conclusion from this experiment is that the EpCAM cells do not have immunosuppressive capability.

Taken together, both Umix and Expanded Hepatocytes are immunogeneic as they stimulated a significant amount of T cells proliferation above the background response to autologous PMBCs. However, Expanded Hepatocytes are significantly more immunogeneic than Umix cells and they directly stimulated purified T cells suggesting that these cells (or a subpopulation of these cells) can function as antigen presenting cells. In contrast, Umix cells stimulated purified T cells poorly relative to responder PMBCs, suggesting that Umix cells cannot function as APCs and require APC help from the PBMC population to stimulate a response.

These data suggest that both cell populations (Umix and Expanded Hepatocytes) are likely to induce a T cell response when transplanted to an allogeneic recipient with an intact immune system. The Expanded Hepatocyte population would be expected to elicit a more robust immune response than Umix cell in vivo.

Adult hepatic stem/progenitor cells do not appear to be immunogenic, as evidenced by their failure to stimulate significant amount of T cells proliferation above the background response to autologous PMBCs. As well, adult hepatic stem/progenitor cells are not immunosuppressive, as evidenced by their inability to suppress significant amounts of T cells proliferation above the background response to autologous PMBCs. Finally, the results suggest that adult hepatic stem/progenitor cells (EpCAM positive cells) express no to low levels of MHC molecules (probably no class II) and they are most likely deficient in expression of co stimulatory molecules (CD80, CD86). Thus, adult hepatic stem/progenitor cells (EPCAM positive cells) may be good candidates for allogeneic cell transplantation.

Indeed, adult derived human hepatic stem/progenitor cells, as defined by expression of EpCAM, Alb, and CK19, have utility as an immune-privileged liver cell therapy product. In vivo xenograft survival assays using adult derived human hepatic stem/progenitor cells may be grafted into a conventional (i.e., non-immune-privileged) site, for example, beneath the kidney capsule using established techniques. Where desirable, an identical number of adult derived, mature hepatocytes may be implanted in control animals.

A skin incision may be made into the flank of recipient mice, the muscle wall incised, and the kidney exteriorized. A subcapsular pocket may be created between the kidney and the kidney cortex, and the cells used for the graft placed into the pocket. The kidney can be replaced into the abdominal cavity and the skin closed with clips. In vivo evaluation of graft survival may be assessed by visual inspection using an operating microscope at selected times post implantation. An evaluation of graft appearance (e.g., necrosis) and vascular in-growth can be made over a time course (e.g., 1 hr, 1 day, 7, 14, 21, 28 days).

In addition, histochemical studies for CD45 over a similar time course of frozen sections of grafts placed under the kidney capsule may be additionally performed. The CD45 antigen is a tyrosine phosphatase, also known as the leukocyte common antigen (LCA). CD45 is present on all human cells of hematopoietic origin, except erythroid cells, platelets and their precursor cells. The CD45 molecule is required for T cell and B cell activation and is expressed in at least 5 isoforms, depending on the activation status of the cell. A lack of accumulation of CD45+ cells at the graft site generally indicates a lack of an immune response by the host, supporting the notion that the grafted cells are non-immungenic. This finding may be expected form the transplantation of adult human hepatic stem/progenitor cells. Conversely, the control mice, into which adult derived mature hepatocytes are grafted, are to have indications of a vigorous immune response, as indicated by necrotic grafted cells, a lack of vascular in-growth, and accumulation of CD45+ cells.

In this way, the use of substantially non-immunogenic hepatic stem cells to treat liver dysfunction may alleviate in larger part, or in its entirety, the need for common immunosuppresants. Hepatic stem cells may thus be employed to repopulate diseased and/or dysfunctional livers in mammalian patients and thereby restore, in some part, the lost liver function.

In the laboratory, this capability may be determined using, for example, female Balb/c mice (8 to 12 weeks of age) and injecting them with 1 ml/kg of body weight of a teratogen, such as CCl₄, twice a week for a total of 8 times to induce liver injury. Blood chemistry of alanine transaminase may be conducted on the mice at the conclusion of the treatment period to document liver injury. Elevation of enzyme levels relative to untreated control mice should be assessed before inclusion of the animals in the study. Transplantation of adult human hepatic stem cells into the recipient mice may be performed via intrasplenic injection.

Varying doses of cells may be transplanted with a preferable maximum dose of 10⁶ cells/mouse into cohorts of 10 animals under each condition. Animals can be monitored over a time course for evidence of improved liver function by the following criteria: alanine transaminase, albumin, total protein, cholesterol, and triglycerides. Any animals showing signs of distress during the study should be euthanized, as studies should not be conducted with death as an endpoint.

Thus, groups of five animals under each transplant condition may be euthanized 4 and 16 weeks post transplantation. The following organs may be harvested: liver, kidney, brain, heart, lungs, spleen. Serum specimens may also be obtained for routine blood chemistry and CBC. Similarly, organs may be processed for immunohistochemistry, H&E, and RNA analysis. Specimens are accordingly stained with antibodies to detect markers of the transplanted cells (anti-beta galactosidase antibodies).

Alternatively, FISH analysis for detection of the Y chromosome may be performed to identify the transplanted male cells. Serial sections are monitored for expression of hepatic proteins (in the liver) as well as DAPI staining to determine the ploidy of cells (to evaluate the possibility of cell fusion). Serial sections of spleen, kidney, brain, lung, heart would document evidence of transplanted cells outside the liver.

An alternative approach may involve transplanting adult human hepatic stem cells into ApoE deficient mouse. Briefly, mice homozygous for the Apoetm1Unc mutation show a marked increase in total plasma cholesterol levels that are unaffected by age or sex. Fatty streaks in the proximal aorta are found at 3 months of age. The lesions increase with age and progress to lesions with less lipid but more elongated cells, typical of a more advanced stage of pre-atherosclerotic lesion. Moderately increased triglyceride levels have been reported in mice with this mutation on a mixed C57BL/6×129 genetic background. Aged APOE deficient mice (>17 months) have been shown to develop xanthomatous lesions in the brain consisting mostly of crystalline cholesterol clefts, lipid globules, and foam cells. Smaller xanthomas were seen in the choroid plexus and ventral formix. Recent studies indicate that APOE deficient mice have altered responses to stress, impaired spatial learning and memory, altered long term potentiation, and synaptic damage.

These mice may be used as hosts to transplant adult human hepatic stem cells via an intrasplenic injection into 4-6 week old homozygous B6.129P2-Apoetm1Unc mice. The recipient groups may include 5 animals in each group: Group 1: control—saline injection; no cells; Group 2—1×10⁶ human stem cells; Group 3—2×10⁶ human stem cells; Group 4—5×10⁵ human stem cells, Group 5—1×10⁶ human mature hepatocytes. A mortality rate of ˜15% may be expected with transplant experiments based on historic controls.

End-point analysis performed over a time course following transplantation may include ApoE (analyzed in the serum by western blot analysis at select time points of 2 weeks, 4 weeks, 8 weeks, 12 weeks). The detection of ApoE in these previously deficient mice following transplantation of adult human hepatic stem cells would support the notion that these cells may be used without immunosuppression of the recipient to reconstitute liver function.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

We claim:

1. A method of repopulating the liver compartment of a mammal comprising:
(a) providing isolated non-immunogenic hepatic progenitors; and
(b) introducing said isolated hepatic progenitors into a liver compartment of a mammal, provided that such introduction is carried out in the absence of immune-suppressing amounts of an immunosuppressant.

2. The method of claim 1 in which the isolated hepatic progenitors are introduced by implantation or injection.

3. The method of claim 1 in which a sub-immune-suppressing amount of an immunosuppressant is administered to the mammal.

4. The method of claim 3 in which the sub-immune-suppressing amount is less than about 10 mg of an immunosuppressant per kilogram of the mammal.

5. The method of claim 3 in which the sub-immune-suppressing amount is less than about 5 mg of an immunosuppressant per kilogram of the mammal.

6. The method of claim 3 in which the sub-immune-suppressing amount is less than about 3 mg of an immunosuppressant per kilogram of the mammal.

7. The method of claim 3 in which the sub-immune-suppressing amount is less than about 0.1 mg of an immunosuppressant per kilogram of the mammal

8. The method of claim 3 in which the immunosuppressant is selected from the group consisting of prednisone, azathioprine, cyclosporine, and mycophenolate.

9. The method of claim 1 in which the mammal is a non-human.

10. The method of claim 1 in which the mammal is a human.

11. The method of claim 10 in which the human is an allogenic recipient.

12. The method of claim 11 in which the allogenic recipient is neither immune-compromised nor immune-privileged.

13. The method of claim 1 in which the isolated non-immunogenic hepatic progenitors are further characterized as non-immunosuppressive.

14. The method of claim 1 in which the isolated non-immunogenic hepatic progenitors are positive for EpCAM expression.
